(19) United States
(12) Patent Application Publication
(10) Pub. No.: US 2005/0171558 A1
(43) Pub. Date: Aug. 4, 2005

(54) Neurosurgery targeting and delivery system for brain structures

(76) Inventors: Rony A. Abovitz, Hollywood, FL (US); Arthur E. Quaid III, Hollywood, FL (US); Benny Hagag, Plantation, FL (US); Thomas Hartkens, London (GB)

Correspondence Address: MUNSCH, HARDT, KOPF & HARR, P.C., INTELLECTUAL PROPERTY DOCKET CLERK, 1445 ROSS AVENUE, SUITE 4000, DALLAS, TX, 75202-2790, US

(21) Appl. No.: 10/967,905

(22) Filed: Oct. 18, 2004

Related U.S. Application Data
(60) Provisional application No. 60/512,246, filed on Oct. 17, 2003.

(51) Int. Cl. A61B 19/00; G06K 9/00

(52) U.S. Cl. 606/130; 382/128

(57) ABSTRACT

Morphing or fitting a brain atlas to a diagnostic image data set of the patient, or to a patient registered to the brain atlas, is enhanced using measurements of one or more physical characteristics of the brain taken with the aid of an instrument as it is being inserted into the brain. The measurements are then compared against known physical characteristics of certain brain structures, thus permitting correlation of the measured physical characteristic to a brain structure. The brain atlas then may be morphed or deformed so that the identified brain structure in the atlas is at or near the position at which the measurement was taken, as known from the tracked position of the instrument.

This patent application claims benefit of U.S. provisional patent application Ser. No. 60/512,246, entitled “Neurosurgery Targeting and Delivery System for Brain Structures,” all of which is incorporated herein by reference.

### BACKGROUND OF THE INVENTION

Functional neurosurgical procedures such as DBS (deep brain stimulation) require accurate targeting of small structures, often deep inside the brain. The procedures often require insertion of a catheter, electrode, endoscope or other device deep inside the brain. Navigation of these instruments inside the brain rely on mechanical frames or guides fixed in some manner relative to the patient and preferably also image guided surgery (IGS) systems. IGS systems are able to track the position of an instrument and display its position relative to patient using a representation of the instrument superimposed on an image or graphical representation of the actual patient's brain. The representation is often generated from one or more 3-dimensional diagnostic data sets generated using magnetic resonance imaging (MRI), computed tomography (CT) or other diagnostic imaging modality. The patient and the image are registered with the IGS system that tracks the position of the instrument (and perhaps also the patient) using one of several known techniques.

Current diagnostic imaging modalities such as CT and MRI often provide a poor view, and sometimes no view, of these structures and, thus, they cannot be used for targeting or navigational purposes. To compensate for these problems, standard atlases of the brain have been used to try and define where these structures may be in the diagnostic scans. Overlays to the images then can be constructed showing the location of these brain structures or just the atlas can be used. The variability in anatomy between patients typically does not allow for a perfect fit between a 3-dimensional image or scan of the specific patient's brain and the standard atlas overlay, thus creating errors that could lead to incorrect targeting and navigation. In order to better fit the atlas to the patient, various schemes to morph or fit the atlas to the specific patient image have been attempted.

### SUMMARY OF THE INVENTION

In accordance with one aspect of the invention, morphing or fitting a brain atlas to a diagnostic image data set of the patient, or to a patient registered to the brain atlas, is enhanced using measurements of one or more physical characteristics of the brain taken with the aid of an instrument as it is being inserted into the brain. These measurements are then compared against known physical characteristics of certain brain structures, thus permitting correlation of the measured physical characteristic to a brain structure. The brain atlas then may be morphed or deformed so that the identified brain structure in the atlas is at or near the position at which the measurement was taken, as known from the tracked position of the instrument. Morphing may be based on more than one measurement or measurement location. With better morphing, a surgeon's placement of, for example, a stimulating electrode, drug delivery catheter or other device is more precise, with less error.

In one example of a preferred embodiment of the invention, neuronal microelectrode recording (MER) signals measured by an electrode intra-operatively can be compared to a database of MER signals for know brain structures to determine. MER signals from different structures in the brain possess differentiating characteristics that can be used to correlate the measured MER signal to a brain structure.

In accordance with another aspect of the invention, an electrode having an extendable micro-electrode or an array of extendable micro-electrodes permits correction of small targeting errors and may enable identification of key target structures or areas in a patient's brain with only one electrode pass. Like branches extending from a trunk of a tree, the micro-electrodes may extend out in many different directions, from many different points along the main electrode.

### DETAILED DESCRIPTION

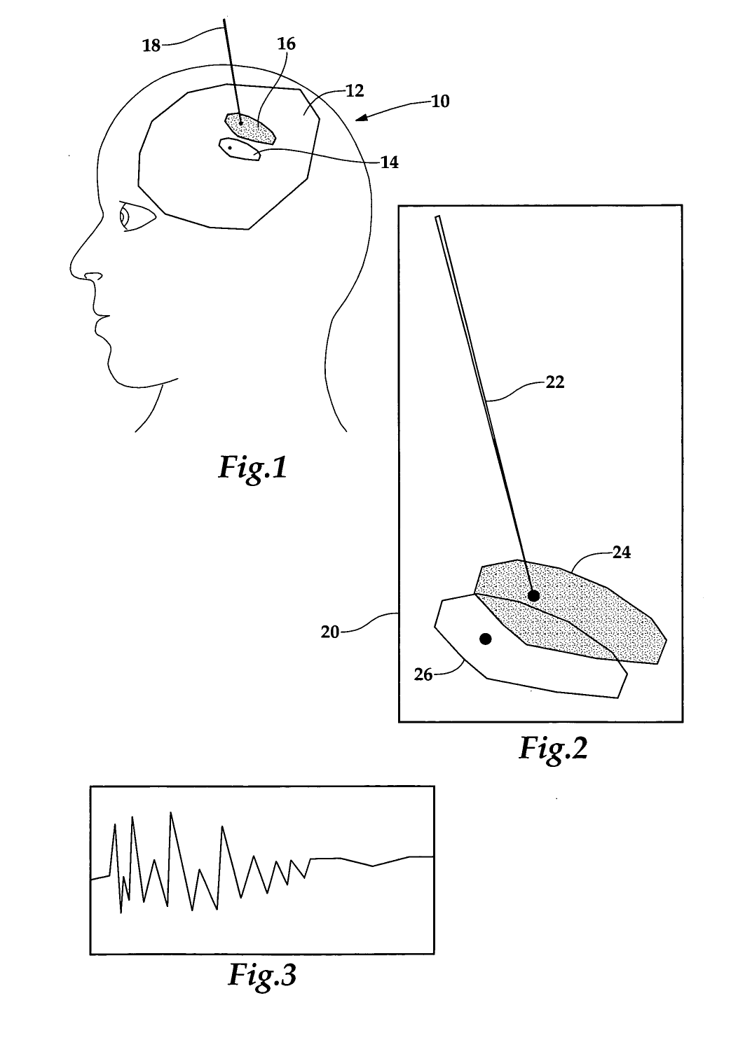

FIG. 1 is a schematic illustration of a patient's head 10, his brain 12, two brain structures 14 and 16, and an electrode 18 that is being inserted into the brain. FIG. 2 is a schematic illustration of a representative example of a display 20 of a surgical navigation system or of a computer running planning software displaying graphic representation 22 of an electrode being inserted into the brain and of target area 24 of the brain as estimated using a brain atlas. These representations may be overlaid onto images generated from diagnostic image data sets taken of the patient (not shown). A graphical representation 26 of the actual or true position of target area in the patient's brain is also indicated simply to illustrate one problem being addressed.

FIG. 3 is a representative recording (not intended to be true) of electrical activity in a patient's brain as detected or measured by the electrode (or microelectrode) inserted into a patient's brain. This signal may be shown on a display of a surgical navigation system.

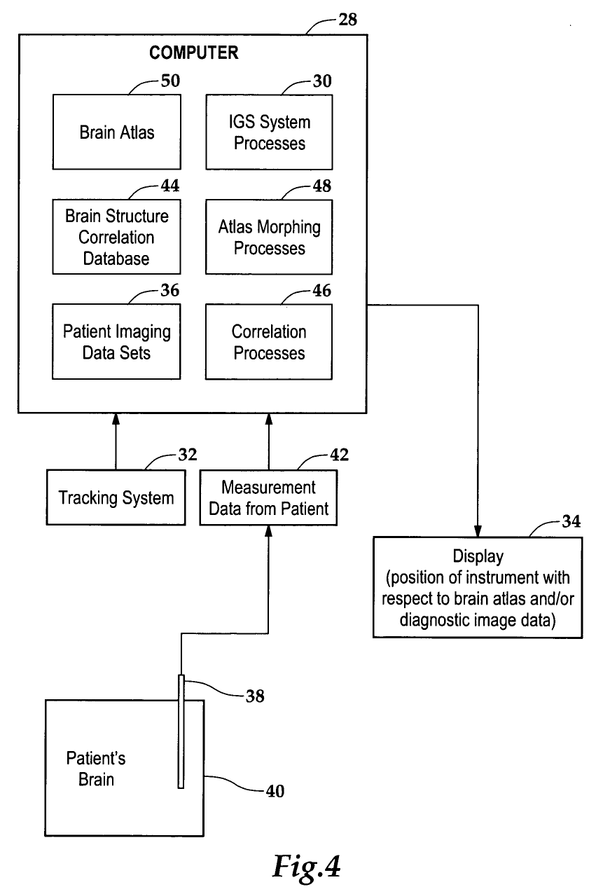

Referring to FIG. 4, image guided surgery systems, also called surgical navigation systems, are well known. An example of such a system, illustrated by FIG. 4, includes IGS processes 30 running one programmable computer, such as computer 28, a tracking system 32 and a display monitor 34. The tracking system, also sometimes called a localizer, is able to locate in three dimensions the position of certain objects within its field view (or within its proximity). In the present example, it is used to locate and track the position of an electrode or other instrument being inserted into a patient's brain. References to the tracking system may, depending on the context, also include computer-based processes associated with identifying, locating and tracking an object, which are collected for purposes of the illustration into the IGS processes 30. The tracking system may also be a passive, semi-active, or active physical robotic device that is physically coupled to the object of interest and can determine its position. The coordinates of identified objects within the field of view are passed to other IGS processes that use them. The details of operation of such systems are well known and will not be repeated here. Patient imaging data sets, such as an MRI or CT scan data sets, or derivatives of them, are also preferably stored for access by the IGS system. For purposes of this example only, they are stored on computer 28. They could also be stored, for example, on a network.

A surgeon plans the location of his target within a patient's brain initially based on the best prediction of the surgical navigation system. The surgeon's planning may include standard anterior commisure (AC) and posterior commisure (PC) line planning. It may also include a brain atlas capable of morphing to the patient specific anatomy. After the patient's anatomy, the diagnostic imaging data set 36 and brain atlas 50 are registered (using known procedures), the surgeon then places an instrument 38 into the patient's anatomy, i.e., brain 40, to obtain data on predetermined physical characteristics for verifying the actual structure. Measured data 42 from the patient is transmitted to, for example, computer 28 and correlated with brain structure correlation database 44 using correlation processes 46. This preferably done on a continual, frequent or periodic (but not necessarily consistent) basis. Morphing processes 48 then correlate brain atlas structure 50, which was used for planning and/or navigation, to the structure or area of the brain predicted by the brain structure correlation data based on the measured data, and updates the position, size, and/or shape of the representation of the brain structure in the atlas based on this data. This updated information is then graphically displayed on monitor 34 for the surgeon to see and use for planning and navigation.

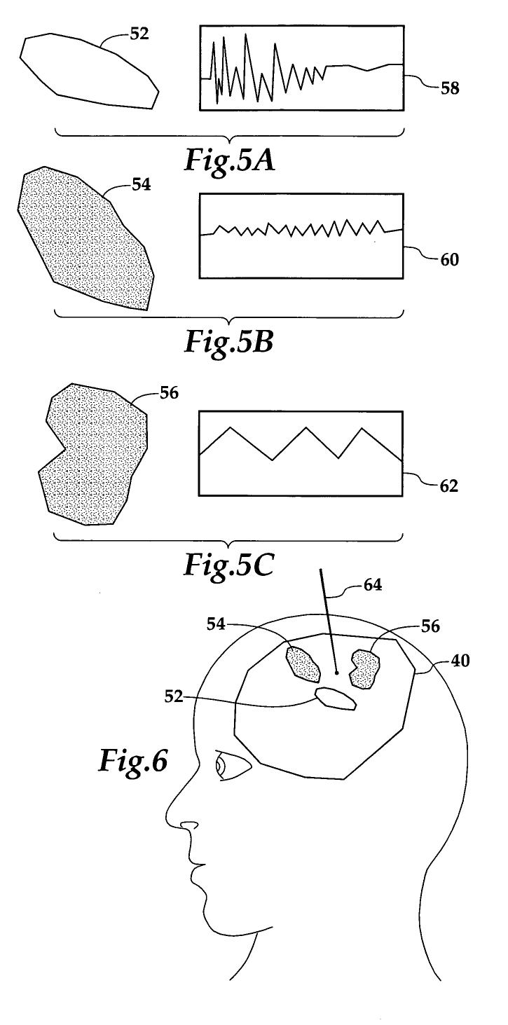

In one example, micro-electrode recording (MER) signals may serve, for example, as the measured physical characteristic of the brain used to make a correlation between the location of the instrument and a brain structure for purposes of morphing or better fitting a brain atlas to a particular patient and/or a diagnostic image data set of the patient. However, although MER may have certain advantages, other types of sensors capable of identifying or detecting variations in tissue structure, anatomy, physiology, or other specific characteristics could also be used. Examples include signals from optical viewers and micro MRI. As illustrated by FIGS. 5A, 5B, 5C and 6, different brain structures (or areas of the brain) 53, 54, and 56 have associated with them different MER signals 58, 60 and 62, respectively. The illustrated MER signals are intended to be representative and do not represent true MER signals. These differences can be exploited to differentiate between different areas of the brain as a diagnostic electrode 64 is being inserted into the brain. Representative MER signals for different structures or areas of the brain are stored in brain structure correlation database 44 to act as references for comparison to actual or measured MER signals. Database 44 is intended to represent a collection of stored information, and thus can represent any store of data, regardless of form. It may comprise, for example, multiple databases or distributed databases, and may be stored in computer memory or in any other type of media. For purposes of the following discussion, this data will be referred to as the brain structure neuronal recording database (BSNRD).

Depending on the type of analysis employed to make the comparison, the reference MER signals may be stored in a number of different forms. For example, actual waveforms or one or more parameters that represent components or characteristics of the signal of the signal, which are significant for differentiating areas or structures of the brain, could be stored in the database. References herein to representative or reference MER signals or MER data are intended to include parameters, characteristics or other representations describing the actual waveforms or components of it, unless otherwise specified. The BSNRD also includes associations between the reference MER signals and the location/topology of the corresponding anatomical structure. The BSNRD is not limited to any particular type of data structure, and could include multiple different data structures, depending on the particular implementation.

While the electrode is moved along its planed path through the brain, the MER data is continuously or periodically compared with the reference MER signals in the BSNRD. Since the location of the tip of the electrode is known as a result of the tracking system 32 locating the visible portion of the electrode, comparisons or correlations can be limited, if desired, to a subset of brain structures in general proximity to the electrode, assuming that the subject brain is not abnormal.

It is possible that the only reference MER signals are available for structures that are interesting for the particular application (e.g., DBS targets and their surrounding structures). If so, the actual MER data from the patient may not be matched or correlated with any reference MER data in the database as the tip of the electrode passes through “non-critical” regions in the brain along its path to a target.

When the MER signal measured on the patient begins to match stored reference MER signal or data, the position of the tip of electrode represents the surface of the “matched” structure in the database. If such electrode's position does not correspond to the position of any point on the surface of the structure, then we know that the brain atlas (represented by the structures in the database) does not correspond with the current position of the electrode. In order to update the brain atlas, the point on the structure's surface corresponding to the current position of the electrode also needs to be known. As there is not enough information to identify unambiguously this point, the point on the structure's surface that is closest to the position of the electrode can be taken. This point-to-surface correspondence can be used to update or morph the brain atlas according to the current electrode position and its MER signal. While the procedure is continued, more and more point-to-surface correspondences are established. Each of the brain atlas updates or registrations preferably take into account all known correlations in order to converge to the best fit.

In addition, even when the MER is not near the boundary of a structure, it provides information, as described above, as to which structure the tip is sensing. However, due to the errors described above, the brain atlas may indicate that the tip is outside of the sensed structure. In that case, the brain atlas can be updated or morphed such that the sensed structure in the atlas includes the current electrode position.

Another example of a physical characteristic that can be used as a reference is data generated through micro-imaging techniques. As an alternative to using an electrode and MER signals, a micro imaging system, optical sensor (probe) capable of reading optical signals in the brain tissue, or an electrical sensor capable of reading the specific tissue electrical characteristics resistance/conductivity, and others could be used. MRI, ultrasound or other type of micro-imaging device is placed at the tip of a probe or catheter and inserted into the brain tissue. The micro-imaging catheter generates an image of a volume around it. This image is then compared to known images of the patient's brain structures and the brain atlas is morphed based on the identified brain structure and the known position of the micro-imaging system (known from the position of the probe or catheter), just as actual MER data is compared to reference MER data. The micro-imaging data could also be compared to the patient's pre-operative diagnostic image data set to measure brain shift between the MRI scan and the patient during the surgery, and then compensate for it in the IGS processes.

There is no technical limitation on the number of passes that could be made with the instrument to gather the data to morph the brain atlas. With the brain atlas fitting better the patient's actual anatomy, a surgeon is able to more precisely place a stimulating electrode, drug delivery catheter, or other device, with less error.

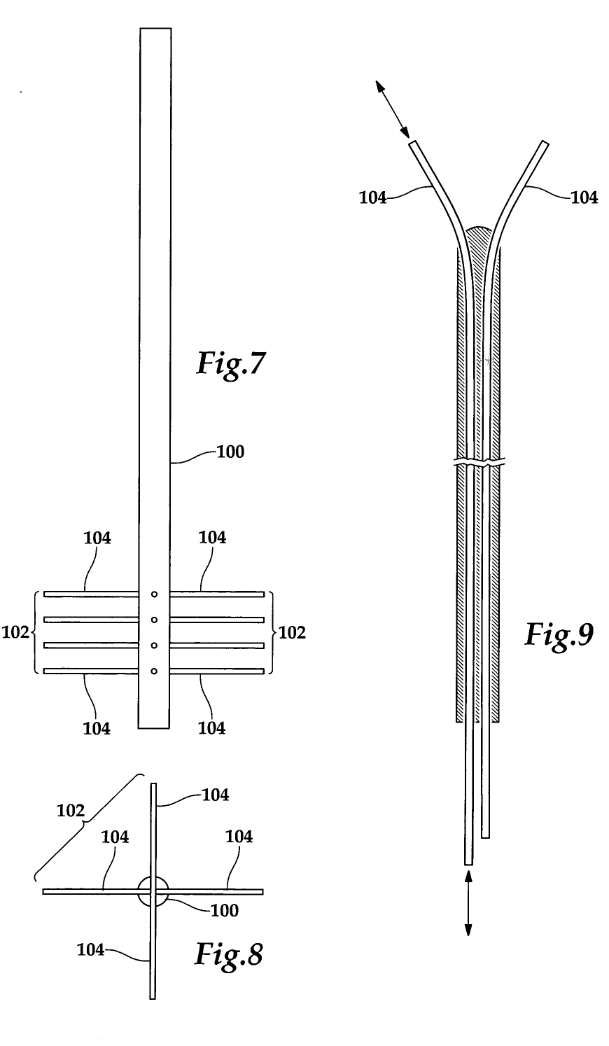

FIGS. 7 and 8 schematically illustrate an electrode 100 with an extendable and retractable micro-array 102. FIG. 9 shows a cross-section view of an alternative embodiment of the electrode with extendable and retractable micro-electrodes. The electrode is similar to a standard neuronal recording electrode, but allows a surgeon to open the array of mini-electrodes 104 into a local target region in the brain. In the event that the initial trajectory of the electrode as placed in the brain is not on target, which is very likely, the surgeon is able to open the array to cover a broader region of the brain. The array is opened in a manner that tends to reduce or minimize damage to the surrounding brain tissue. Each microelectrode is pushed into the brain tissue along a linear (curved or straight) trajectory to reduce tearing, as illustrated by FIG. 9. Readings from each micro-electrode can be recorded and tagged in addition to readings from the trunk. This array of readings can then be used in connection with the system and processes described in connection with FIGS. 1-6, which then can better morph the brain atlas and targeting scheme into the exact location of the true anatomic target.

One advantage of this feature (alone or coupled to the other features) is that it can enable the identification of key targets in the brain with only one electrode pass. A second advantage is the ability of the electrode to become a permanently implantable device for neurostimulation, which may allow for the stimulation of many different regions in the brain, or different brain structures.

The electrode may also be adapted to permit delivery of a drug or a biological therapy, a gene or virus vector, for example. A separate stimulator/delivery control module may control the amount of electrical current and/or drug/biotech therapy delivered. The delivery can occur through internal channels, or through adjacent channels.

The electrode (with or without additional channels to deliver a drug or a biological therapy, a gene or virus vector, for example) may also continuously or periodically be transmitting signals to a computing device (external or implantable) which is connected to the electrode. The computing device may use the incoming signals from the electrode in one or more algorithms to update the target positions if the patient physiology is changing, or to modulate the amount of electrical and/or drug and/or biological therapy and/or gene and/or virus vector. The amount of therapy to be delivered can be based completely on the incoming signals(s), or the incoming signal(s) may be used as part of an algorithm(s) to determine the appropriate dosage for that specific patient. The patient dosage algorithms may be based on an internally stored or programmed database(s). The dosage algorithm may be affected by variables (not limited to) such as patient height, weight, age, sex, disease, disease location, and disease stage.

The electrode and/or the array of microelectrodes may be coated with various agents which either attract, or repulse the surrounding neuronal tissue. The coating on the electrode and/or the array of microelectrodes may be configured in various patterns to create specific in-growth and/or repulsion pathways for the surrounding neuronal tissue.

The array of microelectrodes, and the positions they take in the patient's brain tissue may also be computer controlled and based on incoming microelectrode neuronal readings, and/or additional local sensing (such as ultrasound and MRI). The pathways of the microelectrodes may be controlled by a computer to follow along specific gradients of signal, or signal trends.

1. A method for morphing or fitting a brain atlas to a diagnostic image data set of the patient, or to a patient registered to the brain atlas, the method comprising
receiving measurements indicative one or more physical characteristics of the brain taken with the aid of an instrument as it is being inserted into the brain;
tracking the position of the instrument;
comparing the measurements against known physical characteristics of certain brain structures, thereby correlating of the measured physical characteristic to a brain structure; and
morphing or deforming the brain at last ed or deformed so that the identified brain structure in the atlas is at or near the position at which the measurement was taken.

2. The method of claim of claim 1, wherein the instrument is comprised of an electrode having one or more selectively extendable micro-electrodes.

3. Computer readable memory storing a computer program which, when read and executed by a computer, causes the computer to undertake the following:
receiving measurements indicative of one or more physical characteristics of the brain taken with the aid of an instrument as it is being inserted into the brain;
tracking the position of the instrument;
comparing the measurements against known physical characteristics of certain brain structures, thereby correlating of the measured physical characteristic to a brain structure; and
morphing or deforming the brain at last ed or deformed so that the identified brain structure in the atlas is at or near the position at which the measurement was taken.

4. A surgical navigation system, comprising:
a localizer for tracking the position of an instrument; and
a computer in communication with the localizer for receiving information from which to determine the position of an instrument;
wherein the computer stores a computer program that, when executed causes the computer to undertake the following process:
receiving measurements indicative one or more physical characteristics of the brain taken with the aid of an instrument as it is being inserted into the brain;
tracking the position of the instrument;
comparing the measurements against known physical characteristics of certain brain structures, thereby correlating of the measured physical characteristic to a brain structure; and
morphing or deforming the brain at last ed or deformed so that the identified brain structure in the atlas is at or near the position at which the measurement was taken.

5. An instrument for probing the brain, comprising an electrode and a plurality of retractable and extendable microelectrodes.
